(19) United States
(12) Patent Application Publication
(10) Pub. No.: US 2020/0397953 A1
(43) Pub. Date: Dec. 24, 2020

(54) INJECTABLE SHEAR-THINNING HYDROGELS AND USES THEREOF

(71) Applicants: Massachusetts Institute of Technology, Cambridge, MA (US); The Brigham and Women's Hospital, Inc., Boston, MA (US)

(72) Inventors: Robert S. Langer, Newton, MA (US); Carlo Giovanni Traverso, Newton, MA (US); Jinyao Liu, Shanghai (CN); Yan Pang, Shanghai (CN)

(73) Assignees: Massachusetts Institute of Technology, Cambridge, MA (US); The Brigham and Women's Hospital, Inc., Boston, MA (US)

(21) Appl. No.: 16/857,279

(22) Filed: Apr. 24, 2020

Related U.S. Application Data
(60) Provisional application No. 62/865,472, filed on Jun. 24, 2019.

(51) Int. Cl. A61L 31/14; A61L 31/04; A61L 31/02

(52) U.S. Cl. CPC A61L 31/145; A61B 2017/00818; A61L 31/028; A61L 31/042

(57) ABSTRACT

The present disclosure provides methods of forming interlayer tissue cushions (e.g., submucosal cushions) using shear-thinning hydrogels comprising an anionic polysaccharide and layered silicate and subsequent use of the cushions for removing protrusions (e.g., lesions, such as polyps or tumors) from above the cushions. Further provided are uses, methods of treatment, and kits.

### RELATED APPLICATIONS

The present application claims priority under 35 U.S.C. § 119(e) to U.S. provisional application, U.S. Ser. No. 62/865,472, filed Jun. 24, 2019, which is incorporated herein by reference.

### GOVERNMENT SUPPORT

This invention was made with Government support under Grant No. R01 EB000244 awarded by the National Institutes of Health (NIH). The Government has certain rights in the invention.

### BACKGROUND OF THE INVENTION

Polypectomy remains the single intervention facilitating the interruption of polyp progression towards cancer. Approximately over 15 million colonoscopies are performed annually in the United States with ˜20-25% of these involving polypectomies many of which are performed with the aid of a submucosal injection to facilitate resection^{[1-3]} Endoscopic mucosal resection is a commonly used minimally invasive technique applied in the removal of large polyps (≥2 cm) and early stage tumors because of its simplicity and safety.^{[4-5]} This is often assisted through an initial submucosal injection used for the establishment of a cushion between the surface mucosa and muscular tissue layers. Since its description in 1984,^{[6]} normal saline (0.9 wt % sodium chloride) has been the main injection fluid used for endoscopic mucosal resection. Recently, other fluids including hypertonic saline, hypertonic dextrose water, autologous blood, sodium hyaluronate, glycerol, hyaluronic acid, succinylated gelatin, hydroxypropyl methylcellulose, poloxamer, and fibrinogen have been applied to prolong cushion stability by increasing the viscosity of the fluid.^{[7-13]} However, the application of these solutions has been largely restricted by unmet safety profiles and durations. Specifically, the heights of cushions elevated by hypertonic saline, dextrose water, and glycerol reduce to less than 50% in 30 mins.^{[14-16]} Additionally, injection solutions showing prolonged duration can be associated with administration challenges. For example, carboxymethylcellulose solutions, can require a special 18 gauge submucosal injection needle catheter to minimize injection resistance because of its high viscosity.^{[17,18]} Moreover, hyaluronic acid potentially stimulates the growth of residual tumor tissues.^{[19]} Fibrinogen and autologous blood are biologic materials which may increase the risk of infection via contamination.^{[20,21]} Submucosal injection solutions play a role in the successful, safe, and intact removal of lesions as they not only lift up diseased mucosa but also provide a gap between the mucosal and deeper layers of tissues facilitating the resection of lesions. Ensuring complete, safe resection mitigates the risk of local recurrence.^{[22]} Hence, an ideal injection solution for submucosal elevation should be biocompatible, easily injectable, and/or provide durable (e.g., longer lasting) submucosal cushions.

### SUMMARY OF THE INVENTION

One potential set of materials with enhanced biocompatibility and prolonged duration of lift are hydrogels due to their high-water content and stiffness, which can simultaneously minimize toxicity and resist diffusion.^{[23]} Unfortunately, conventional hydrogels crosslinked by chemical bonds or physical interactions are generally not amenable to injection through an endoscopic needle.^{[24]} In situ formation of hydrogels is an effective approach to solve this problem and has been widely used to facilitate in vivo application of hydrogels. However, the formation of hydrogels by in situ chemical reactions requires the injection of two or more components simultaneously,^{[25]} which is challenging for endoscopic submucosal injection. Additionally, the physical crosslinking of hydrogels, such as thermo-triggered gelation, has the risk of obstructing the endoscopic needle during injection.^{[26,27]}

Described herein is the development of endoscopically injectable shear-thinning hydrogels (EISHs) that can be easily injected through an endoscopic needle and immediately recover their mechanical properties as a solid gel upon deployment in the submucosal compartment. These gels demonstrate their potential to serve as safe and easily injectable agents that can provide durable submucosal cushions in a subject. Given their unique characteristics, EISHs are useful in endoscopic mucosal resection technique for removal of polyps and early stage tumors.

In one aspect, the disclosure provides methods of forming a submucosal cushion comprising injecting into the submucosa an effective amount of a shear-thinning hydrogel.

In one aspect, the disclosure provides methods of separating portions of a tissues comprising injecting between tissue planes an effective amount of a shear-thinning hydrogel

In certain aspects, the shear-thinning hydrogel comprises a layered silicate (e.g., Laponite®) and an anionic polysaccharide (e.g., alginate). In certain aspects, the shear-thinning hydrogel is injectable, even through a high gauge needle (e.g., a 25 gauge needle, an endoscopic needle). In some aspects, the properties of the shear-thinning hydrogel are tunable based on the concentration of the components. In certain aspects, the shear-thinning hydrogels form non-toxic, stable, and/or long-lasting hydrogels when compared to saline. In certain aspects, the shear-thinning hydrogel remains a liquid and easily flows until injection, at which point the shear-thinning hydrogel forms a solid gel.

In another aspect, the disclosure provides methods of removing a lesion comprising injecting into the submucosa under the lesion (e.g., polyp, precancerous lesion, cancerous lesion) an effective amount of a shear-thinning hydrogel comprising a layered silicate and an anionic polysaccharide (e.g., a shear-thinning hydrogel comprising sodium alginate and Laponite®) and resecting the lesion.

Also provided herein are methods of preventing or treating cancer comprising injecting into the submucosa under a tumor an effective amount of a shear-thinning hydrogel comprising a layered silicate and an anionic polysaccharide (e.g., a shear-thinning hydrogel comprising sodium alginate and Laponite®) and resecting the lesion or tumor.

In some aspects, the disclosure provides shear-thinning hydrogels for use in forming a submucosal cushion by injecting into the submucosa an effective amount of a shear-thinning hydrogel comprising a layered silicate and an anionic polysaccharide (e.g., a shear-thinning hydrogel comprising sodium alginate and Laponite®).

The present disclosure further provides shear-thinning hydrogels for use in removing a lesion comprising injecting into the submucosa under the lesion an effective amount of a shear-thinning hydrogel comprising a layered silicate and an anionic polysaccharide (e.g., a shear-thinning hydrogel comprising sodium alginate and Laponite®) and resecting the lesion.

Also provided by the disclosure are kits. In certain aspects, the kits comprise a layered silicate and an anionic polysaccharide, and optionally, water. In some aspects, the kits comprise Laponite®, sodium alginate, and optionally, water. In certain aspects, the kits further comprise instructions for use and/or supplies needed for injection/delivery.

The details of certain embodiments of the invention are set forth in the Detailed Description of Certain Embodiments, as described below. Other features, objects, and advantages of the invention will be apparent from the Definitions, Examples, and Claims.

### BRIEF DESCRIPTION OF THE DRAWINGS

FIGS. 1A to 1D show a endoscopically injectable shear-thinning hydrogel (EISH) platform and preparation approach.

FIG. 1A shows a schematic illustration of exfoliated clay nanosheets by the interaction of their positive charged edges with anionic sodium alginate.

FIG. 1B shows the preparation of EISHs by dispersing clay nanosheets in 0.2 wt % sodium alginate.

FIG. 1C shows TEM images of the exfoliated clay nanosheets.

FIG. 1D shows the feasibility of injecting EISHs through a 25-gauge needle and the immediate reformation of a stable gel after injection.

FIGS. 2A to 2D show the rheological properties of EISHs where G′ (filled symbols) and G″ (empty symbols) represent storage and loss modulus, respectively.

FIG. 2A shows oscillatory time sweeps of EISHs. Sweeps were performed at 0.5% strain and 6.3 rad s⁻¹.

FIG. 2B shows oscillatory frequency sweeps of EISHs. Sweeps were performed at 0.5% strain.

FIG. 2C shows oscillatory strain sweeps of EISHs. Sweeps were performed at 6.3 rad s⁻¹.

FIG. 2D shows deformation and recovery of EISHs. Gels evolved over time from repeated cycles of 3 min low 0.5% strain and 2 min high 500% strain oscillations at 6.3 rad s⁻¹.

FIGS. 3A to 3E show the in vitro evaluation of EISHs where error bars show standard deviation (n=3).

FIG. 3A shows a photo of an endoscopic needle.

FIG. 3B shows fluent injection of EISHs via the endoscopic needle.

FIG. 3C shows the storage modulus of EISHs before and after injection by a 25-gauge needle at an injection speed of 0.25 mL s⁻¹.

FIG. 3D shows gel restoration kinetics of EISHs subjected to shear force induced by syringe injection.

FIG. 3E shows gel erosion kinetics of EISH cushions in saline at 37° C.

FIGS. 4A to 4F show the endoscopic development of submucosal cushions.

FIG. 4A shows a pig colon before submucosal injection of 1.5 cc of 2 mg mL⁻¹ EISH.

FIG. 4B shows a pig colon after submucosal injection of 1.5 cc of 2 mg mL⁻¹ EISH

FIG. 4C shows endoscopic images of the submucosal cushions developed by saline at 0 minutes post-injection, showing the prolonged duration of the cushions developed by EISHs.

FIG. 4D shows endoscopic images of the submucosal cushions developed by saline at 1 minute post-injection, showing the prolonged duration of the cushions developed by EISHs.

FIG. 4E shows endoscopic images of the submucosal cushions developed by 2 mg mL⁻¹ EISH at 0 minutes post-injection.

FIG. 4F shows endoscopic images of the submucosal cushions developed by 2 mg mL⁻¹ EISH at 3.5 minutes post-injection, showing the prolonged duration of the cushions developed by EISHs.

FIGS. 5A to 5D show in vivo submucosal cushion duration. The cushions were lifted by 2 cc of saline (top right), 1 mg mL¹ EISH (bottom right), 2 mg mL⁻¹ EISH (bottom left), and 3 mg mL⁻¹ EISH (top left).

FIG. 5A shows a photograph of submucosal cushions in pig colon post-injection at 0 minutes.

FIG. 5B shows a photograph of submucosal cushions in pig colon post-injection at 30 minutes.

FIG. 5C shows a photograph of submucosal cushions in pig colon post-injection at 60 minutes.

FIG. 5D shows a photograph of submucosal cushions in pig colon post-injection at 120 minutes.

FIG. 5E shows the duration of cushions lifted by 2 cc of EISHs with different concentrations in pig colon. Error bars show standard deviation (n=3).

FIG. 5F shows the relationship between time post-injection and height of cushions developed by 2 mg mL⁻¹ EISHs with different volumes. Error bars show standard deviation (n=3).

FIG. 5G shows the duration of cushions lifted by 2 cc of EISHs with different concentrations in small intestine. 2 cc of saline solution was injected as a control. Error bars show standard deviation (n=3).

FIGS. 6A to 6F show the in vivo toxicity of saline and EISHs.

FIG. 6A shows the H&E staining image of pig colon without any treatment. The tissues were harvested at 2 h post-injection.

FIG. 6B shows the H&E staining image of pig colon with submucosally injected with saline. The tissues were harvested at 2 h post-injection.

FIG. 6C shows the H&E staining image of pig colon with submucosally injected with 3 cc of 3 mg mL⁻¹ EISH. The tissues were harvested at 2 h post-injection.

FIG. 6D shows the H&E staining images of pig colon without any treatment on the top of the mucus for 2 h

FIG. 6E shows the H&E staining images of pig colon incubated with saline on the top of the mucus for 2 h.

FIG. 6F shows the H&E staining images of pig colon incubated with 3 cc of 3 mg mL⁻¹ EISH on the top of the mucus for 2 h.

FIG. 7A shows a top view of ex vivo cushion development in pig colon by injection of EISHs.

FIG. 7B shows a side view of ex vivo cushion development in pig colon by injection of EISHs.

### DEFINITIONS

Unless otherwise required by context, singular terms shall include pluralities, and plural terms shall include the singular.

The language “in some embodiments” and “in certain embodiments” are used interchangeably.

The following definitions are more general terms used throughout the present application:

The singular terms “a,” “an,” and “the” include plural references unless the context clearly indicates otherwise. Similarly, the word “or” is intended to include “and” unless the context clearly indicates otherwise.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

When a range of values (“range”) is listed, it is intended to encompass each value and sub-range within the range. A range is inclusive of the values at the two ends of the range unless otherwise provided.

The term “salt” refers to ionic compounds (e.g., polysaccharide) that result from the neutralization reaction of an acid and a base. A salt is composed of one or more cations (positively charged ions (e.g., Na⁺, Li⁺, K⁺, Mg²⁺, Ca²⁺)) and one or more anions (negative ions (e.g., on a negative charge on a polysaccharide)) so that the salt is electrically neutral (without a net charge). Salts of the disclosure include those derived from inorganic and organic acids and bases. Examples of salts (e.g., pharmaceutically acceptable and nontoxic) salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvent” refers to a substance that dissolves one or more solutes, resulting in a solution. A solvent may serve as a medium for any reaction or transformation described herein. The solvent may dissolve one or more reactants or reagents in a reaction mixture. The solvent may facilitate the mixing of one or more reagents or reactants in a reaction mixture. The solvent may also serve to increase or decrease the rate of a reaction relative to the reaction in a different solvent. Solvents can be polar or non-polar, protic or aprotic.

The term “toxic” refers to a substance showing detrimental, deleterious, harmful, or otherwise negative effects on a subject, tissue, or cell when or after administering the substance to the subject or contacting the tissue or cell with the substance, compared to the subject, tissue, or cell prior to administering the substance to the subject or contacting the tissue or cell with the substance. In certain embodiments, the effect is death or destruction of the subject, tissue, or cell. In certain embodiments, the effect is a detrimental effect on the metabolism of the subject, tissue, or cell. In certain embodiments, a toxic substance is a substance that has an LD₅₀ of not more than 1,000 milligrams per kilogram of body weight when administered by continuous contact for 24 hours (or less if death occurs within 24 hours) with a test subject (e.g., rat, rabbit) weighing between two and three kilograms, inclusive. The term “nontoxic” refers to a substance that is not toxic.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird. The non-human animal may be a male or female at any stage of development. The non-human animal may be a transgenic animal or genetically engineered animal.

The term “administer,” “administering,” or “administration” refers to implanting, absorbing, ingesting, injecting, inhaling, or otherwise introducing a shear-thinning hydrogel described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a medical history of symptoms). Treatment may also be continued after symptoms have resolved, for example, to delay and/or prevent recurrence of the disease or disorder. In other embodiments, treatment is accomplished by removing a lesion or polyp that could become cancerous. In some embodiments, treatment is accomplished by removing a cancerous lesion.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population of subjects. In some embodiments, prevention is accomplished by removing a lesion or polyp before it becomes cancerous.

The terms “condition,” “disease,” and “disorder” are used interchangeably.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, diseases associated with angiogenesis, inflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, adenocarcinoma; adrenal gland cancer; anal cancer; appendix cancer; biliary cancer (e.g., cholangiocarcinoma); bladder cancer; carcinoid tumor; cervical cancer (e.g., cervical adenocarcinoma); choriocarcinoma; colorectal cancer (e.g., colon cancer, rectal cancer, colorectal adenocarcinoma); connective tissue cancer; epithelial carcinoma; endometrial cancer (e.g., uterine cancer, uterine sarcoma); esophageal cancer (e.g., adenocarcinoma of the esophagus, Barrett's adenocarcinoma); gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; mesothelioma; neuroblastoma; neuroendocrine cancer (e.g., gastroenteropancreatic neuroendoctrine tumor (GEP-NET), carcinoid tumor); ovarian cancer (e.g., cystadenocarcinoma, ovarian embryonal carcinoma, ovarian adenocarcinoma); pancreatic cancer (e.g., pancreatic andenocarcinoma, intraductal papillary mucinous neoplasm (IPMN), Islet cell tumors); penile cancer (e.g., Paget's disease of the penis and scrotum); plasma cell neoplasia; intraepithelial neoplasms; prostate cancer (e.g., prostate adenocarcinoma); rectal cancer; small bowel cancer (e.g., appendix cancer); small intestine cancer; testicular cancer (e.g., seminoma, testicular embryonal carcinoma); urethral cancer; and vaginal cancer.

The terms “non-neoplastic,” “non-neoplastic mass,” or “non-neoplastic lesion” refers to tissue changes that are pathological but not cancerous. A non-neoplastic mass encompasses a broad assortment of tissue alterations including congenital, degenerative, inflammatory, adaptive, and reparative changes. Some non-neoplastic masses occur normally with age; exposure to a test chemical may either increase or decrease the incidence and/or severity of these spontaneously occurring “background” lesions. In other cases, exposures may induce novel non-neoplastic changes. Some non-neoplastic lesions may progress to tumor formation with time or continued chemical exposure.

An “effective amount” of a composition described herein refers to an amount sufficient to elicit the desired biological response. An effective amount of a composition described herein may vary depending on such factors as the desired cushion height or size. In certain embodiments, an effective amount is a therapeutically effective amount.

A “hydrogel” is a three-dimensional network consisting of cross-linked polymer chains, which form matrices with high water content. A hydrogel may be a hydrophilic network. Hydrogels may exhibit rheological solid-like properties. Hydrogels may also possess a degree of flexibility similar to natural tissue, due to their water content.

The term “shear-thinning” is in rheology to describe non-Newtonian fluids which demonstrate viscous flow under shear stress and subsequent recovery upon removal of the stress. The term “shear-thinning” is normally associated with an effect where a fluid's viscosity (the measure of a fluid's resistance to flow) decreases with an increasing rate of shear stress.

The term “shear-thinning hydrogel” is a hydrogel capable of self-assembling into a gelled network by interaction of its associated non-covalent linkages. A shear-thinning hydrogel may be composed of two or more polymers or oligomers that are held together in unique structural relationships by forces other than those of full covalent bonds. Non-covalent bonding maintains the three-dimensional structure of the hydrogels. There are four commonly mentioned types of non-covalent interactions: hydrogen bonds, ionic bonds, van der Waals forces, and hydrophobic interactions. When subjected to a mechanical shear (such as when forced to flow through a needle, catheter, or cannula), at least some of the non-covalent linkages within the hydrogel disassociate, leading to a disassembly of the gel network and a temporary thinning of the gel (lowering of the viscosity). Upon the removal of the mechanical shear force, the original gel re-assembles/recovers to a state (e.g., viscosity, stiffness, or diffusivity) the same as, or close to, it pre-shear state. The term “shear-thinning hydrogels” are used in the literatures to describe such gels where the recovery of the hydrogel after shear can be nearly instantaneous or be as long as hours.

A “self-healing hydrogel” is a hydrogel which can automatically heal damage and restore itself to normality without the intervention of an external stimuli. A self-healing hydrogel is able to repair the structural damages and recover the original functions.

The term “gel-sol transition” is used to describe a change from a gel state to a liquid state.

The term “polysaccharide” is used to describe long chains of carbohydrate molecules, such as polymeric carbohydrates composed of monosaccharide units bound together by glycosidic linkages.

The term “silicate” is used to describe any compound or material containing an anion comprising silicon and oxygen (e.g., SiO₃, Si₂O₅, SiO₄, Si₂O₇, Si₄O₁₀). Silicate may also refer to anions that comprise other atoms besides silicon and oxygen (e.g., Si(OH)₆).

The term “Laponite” is a synthetic layered silicate manufactured from naturally occurring inorganic mineral sources. Laponite® is a 2-dimensional nanomaterial composed of disk-shaped nanoscale crystals with a high aspect ratio. These disks can strongly interact with various chemical entities (including small molecules, ions, natural or synthetic polymers, inorganic nanoparticles) and may be functionalized and degraded in physiological environments giving rise to non-toxic products.

The term “exfoliate” as used herein is used to describe a process of separating or dividing layered materials (e.g., into individual sheets or discs). Exfoliation increases the surface area of the material.

The disclosure is not intended to be limited in any manner by the above exemplary terms. Additional terms may be defined in other sections of this disclosure.

### DETAILED DESCRIPTION OF CERTAIN EMBODIMENTS

Before the disclosed systems, hydrogels compositions, methods, uses, and kits are described in more detail, it should be understood that the aspects described herein are not limited to specific embodiments, methods, systems, apparati, or configurations, and as such can, of course, vary. It is also to be understood that the terminology used herein is for the purpose of describing particular aspects only and, unless specifically defined herein, is not intended to be limiting.

Submucosal elevation, the process of instilling material in the submucosal space for separation of the surface mucosa and deeper muscularis layer, is a typical aspect of the endoscopic mucosal resection of lesions performed to facilitate lesion removal and/or maximize safety. Submucosal injection has historically been performed with normal saline though this is limited by its rapid dissipation; solutions ideally need to be easily injectable, biocompatible, and provide a long-lasting submucosal cushion with a desirable height. As disclosed herein, endoscopically injectable shear-thinning hydrogels meet these requirements because of their biocompatible components and ability to form a solid hydrogel upon injection. The shear-thinning hydrogels of the present disclosure were evaluated in a large animal model and can serve as submucosal injection fluids for cushion development. Given these unique characteristics, they have broad application in mucosal resection techniques and dissecting tissue planes.

### Methods and Uses

In one aspect, the disclosure provides methods of forming an interlayer tissue cushion comprising injecting between two tissue layers an effective amount of a shear-thinning hydrogel.

In certain embodiments, the interlayer tissue cushion is a submucosal cushion. In some embodiments, the tissue layers are the mucosa and muscularis.

In one aspect, the disclosure provides methods of forming a submucosal cushion comprising injecting into the submucosa an effective amount of a shear-thinning hydrogel.

In another aspect, the disclosure provides a shear-thinning hydrogel for use in forming a submucosal cushion by injecting into the submucosa an effective amount of a shear-thinning hydrogel comprising sodium alginate and Laponite®.

In certain embodiments, the shear-thinning hydrogel comprises a layered silicate and an anionic polysaccharide.

In some embodiments, the layered silicate is a montmorillonite, saponite, hectorite, or subclasses thereof. In certain embodiments, the layered silicate is a layered nanosilicate. In some embodiments, the layered silicate is a synthetic layered silicate. In some embodiments, the layered silicate is a natural layered silicate. In some embodiments, the layered silicate is referred to as a clay. In certain embodiments, the layered silicate is an aluminum/magnesium silicate. In certain embodiments, the layered silicate is a synthetic layered nanosilicate. In certain embodiments the layered silicate is a montmorillonite. In certain embodiments, the layered silicate has a chemical formula of (Na,Ca)_{0.33}(Al,Mg)₂ (Si₄O₁₀)(OH)₂.nH₂O or Na,Ca)_{0.33}(Al,Mg)₂ (Si₄O₁₀)(OH)₂. In some embodiments, the layered silicate is bentonite. In certain embodiments, the layered silicate has the formula Al₂O₃.4 SiO.2 H₂O or Na_{0.33} [Al_{1.67}Mg_{0.33}] Si₄[OH]₂. In certain embodiments, the layered silicate is a saponite. In certain embodiments, the layered silicate has the formula Ca_{0.25}(Mg,Fe)₃((Si,Al)₄O₁₀)(OH)₂.nH₂O or Ca_{0.25}(Mg,Fe)₃((Si,Al)₄O₁₀)(OH)₂. In certain embodiments, the layered silicate is a hectorite. In certain embodiments, the layered silicate has the formula Na_{0.3}(Mg,Li)₃(Si₄O₁₀)(F,OH)₂. In some embodiments, the layered silicate is Laponite®.

In certain embodiments, the concentration of the layered silicate in the shear-thinning hydrogel is between about 1 to about 6 mg mL⁻¹. In some embodiments, the concentration of the layered silicate is between about 2 and about 5 mg mL⁻¹. In certain embodiments, the concentration of Laponite® is between about 1 to about 6 mg mL⁻¹. In some embodiments, the concentration of Laponite® is between about 2 and about 5 mg mL⁻¹. In certain embodiments, the concentration of Laponite® is about 1, about 2, about 3, about 4, about 5, or about 6 mg mL⁻¹. In certain embodiments, the concentration of Laponite® is about 2, about 3, about 4, or about 5 mg mL⁻¹. In some embodiments, the concentration of Laponite® is 1 mg mL⁻¹. In certain embodiments, the concentration of Laponite® is 2 mg mL⁻¹. In some embodiments, the concentration of Laponite® is 3 mg mL⁻¹. In certain embodiments, the concentration of Laponite® is 4 mg mL⁻¹. In some embodiments, the concentration of Laponite® is 5 mg mL⁻¹. In certain embodiments, the concentration of Laponite® is 6 mg mL⁻¹.

In some embodiments, the anionic polysaccharide is an algin, an arabinoxylan, a carrageenan, a furcellaran, a gellan, a gum arabic, a gum ghatti, a gum karaya, a gum tragacanth, an okra gum, a pectic acid, a pectin, a psyllium seed gum, a xanthan, a xylan, a carboxymethylcellulose, or a salt thereof. In certain embodiments, the anionic polysaccharide is a xanthan, hyaluronic acid, heparin, or an algin, or salt thereof. In some embodiments, the anionic polysaccharide is xanthan gum, or salt thereof In some embodiments, the anionic polysaccharide is hyaluronic acid or a salt thereof. In some embodiments, the anionic polysaccharide is heparin or a salt thereof. In certain embodiments, the anionic polysaccharide is formed by incorporating acidic functions into natural polysaccharides. In some embodiments, the anionic polysaccharide is carboxymethylcellulose or a salt thereof In some embodiments, the anionic polysaccharide is pectin or a salt thereof. In certain embodiments, the anionic polysaccharide is pectic acid or a salt thereof. In some embodiments, the anionic polysaccharide is an algin or a salt thereof. In some embodiments, the anionic polysaccharide is sodium alginate.

In some embodiments, the concentration of the anionic polysaccharide in the shear-thinning hydrogel is about greater than 0 to about 2 wt %. In certain embodiments, the concentration of the anionic polysaccharide is about 0.1 to about 1 wt %. In some embodiments, the concentration of sodium alginate in the shear-thinning hydrogel is about greater than 0 to about 2 wt %. In certain embodiments, the concentration of sodium alginate is about 0.1 to about 1 wt %. In some embodiments, the concentration of sodium alginate is about 1 wt %. In some embodiments, the concentration of sodium alginate is about 0.1 wt %. In some embodiments, the concentration of sodium alginate is about 0.2 wt %. In some embodiments, the concentration of sodium alginate is about 0.3 wt %. In some embodiments, the concentration of sodium alginate is about 0.4 wt %. In some embodiments, the concentration of sodium alginate is about 0.5 wt %.

In certain embodiments, the shear-thinning hydrogel comprises Laponite® and an anionic polysaccharide. In certain embodiments, the shear-thinning hydrogel comprises a layered silicate and sodium alginate.

In certain embodiments, the shear-thinning hydrogel comprises Laponite® and sodium alginate. In certain embodiments, the shear-thinning hydrogel further comprises Laponite®, sodium alginate, and water. In some embodiments, the concentration of Laponite® is between 1 to about 6 mg mL⁻¹, and the concentration of sodium alginate is greater than 0 to about 2 wt %. In some embodiments, the concentration of Laponite® is between 2 to about 5 mg mL⁻¹, and the concentration of sodium alginate is greater than 0 to about 1 wt %. In some embodiments, the concentration of Laponite® is about 2 mg mL⁻¹, and the concentration of sodium alginate is about 0.2 wt %. In some embodiments, the concentration of Laponite® is about 32 mg mL⁻¹, and the concentration of sodium alginate is about 0.2 wt %. In some embodiments, the concentration of Laponite® is about 5 mg mL⁻¹, and the concentration of sodium alginate is about 0.2 wt %. In some embodiments, the concentration of Laponite® is about 5 mg mL⁻¹, and the concentration of sodium alginate is about 0.2 wt %.

In some embodiments, the submucosal cushion created by the shear-thinning hydrogel is long lasting (e.g., maintains its volume, increases in volume, maintains its height, and/or maintains a useful height (e.g., a height at least comparable to a cushion created by saline) for at least 15 minutes, at least 30 minutes, at least 45 minutes, at least 1 hour, at least 1.5 hours, or at least 2 hours). In certain aspects, the submucosal cushion created by the shear-thinning hydrogel is resistant to passive diffusion.

In certain embodiments, the volume of the submucosal cushion created by the shear-thinning hydrogel is maintained overtime. In certain embodiments, the volume of the submucosal cushion created by the shear-thinning hydrogel is at least maintained for at least 20 minutes, 30 minutes, 1 hour, 1.5 hours, or 2 hours. In certain embodiments, the volume of the submucosal cushion created by the shear-thinning hydrogel is at least maintained for at least 1.5 hours. In certain embodiments, the volume of the submucosal cushion created by the shear-thinning hydrogel is maintained for at least 2 hours. In certain embodiments, the volume of the submucosal cushion created by the shear-thinning hydrogel is at least maintained for at least 1.5 hour when the concentration of Laponite® is at least 2 mg mL⁻¹.

In some aspects, the volume of the submucosal cushion created by the shear-thinning hydrogel increases over time. In certain embodiments, the volume of the submucosal cushion created by the shear-thinning hydrogel increase over 20 minutes, 30 minutes, 1 hour, 1.5 hours, 2 hours, or 3 hours. In some embodiments, the volume of the submucosal cushion created by the shear-thinning hydrogel increases over 30 minutes. In some embodiments, the volume of the volume of the submucosal cushion created by the shear-thinning hydrogel increases over 1 hour. In some embodiments, the volume of the volume of the submucosal cushion created by the shear-thinning hydrogel increases over 2 hours. In certain embodiments, the volume of the submucosal cushion increases by about 5%, 10%, 20%, 40%, 50%, 60%, 70%, 80%, 90%, or 100% to result in cushion that is about 1.05, 1.1, 1.2, 1.4, 1.5, 1.6, 1.7, 1.8, 1.9, or 2 times its initial volume after approximately 15 minutes, 30 minutes, 1 hour, 2 hours, or 3 hours. In certain embodiments, the volume of the submucosal cushion increases by about 40% to result in a cushion that is about 1.4, 1.5, or 1.6 times its initial volume after about 2 hours. In certain embodiments, the volume of the submucosal cushion created by the shear-thinning hydrogel increases over time when the concentration of Laponite® is at least 4 mg mL⁻¹. In some aspects, the volume of the submucosal cushion created by the shear-thinning hydrogel increases over time due to water absorption.

In some aspects, the submucosal cushion created by the shear-thinning hydrogel changes in height over time. In certain aspects, the submucosal cushion created by the shear-thinning hydrogel decreases in height over time. In some aspects, the submucosal cushion created by the shear-thinning hydrogel retains a useful height over time. In certain aspects, a useful height means a height at least as high as a submucosal cushion created by saline. In certain aspects, a useful height means a height at least high enough to remove a lesion. In some embodiments, the submucosal cushion created by the shear-thinning hydrogel retains a percentage of its original height, wherein the retained height (e.g., the height of the submucosal cushion after injection wherein the height may decrease over time (e.g., the height of the submucosal cushion after seconds, minutes, or hours after injection)) is still useful (e.g., is high enough to remove a lesion). In some embodiments, the submucosal cushion created by the shear-thinning hydrogel retains at least 90% of its height after 5 minutes. In certain embodiments, the submucosal cushion created by the shear-thinning hydrogel retains at least 80% of its height after 10 minutes. In some embodiments, the submucosal cushion created by the shear-thinning hydrogel retains at least 80% of its height after 20 minutes. In some embodiments, the submucosal cushion created by the shear-thinning hydrogel retains at least 80% of its height after 20 minutes. In certain embodiments, the submucosal cushion created by the shear-thinning hydrogel retains at least 75% of its height after 20 minutes, 30 minutes, or 40 minutes. In certain embodiments, the submucosal cushion created by the shear-thinning hydrogel retains at least 80% of its height after 20 minutes, 30 minutes, or 40 minutes. In some embodiments, the submucosal cushion created by the shear-thinning hydrogel retains at least 70% of its height after 30 minutes. In some embodiments, the submucosal cushion created by the shear-thinning hydrogel retains at least 60% of its height after 40 minutes. In certain embodiments, the submucosal cushion created by the shear-thinning hydrogel retains at least 65% of its height after 50 minutes. In some embodiments, the submucosal cushion created by the shear-thinning hydrogel retains at least 50% of its height after 2 hours. In some embodiments, the submucosal cushion created by the shear-thinning hydrogel retains at least 50% of its height after 20 minutes. In some embodiments, the submucosal cushion created by the shear-thinning hydrogel retains at least 50% of its height after 10 minutes, 20 minutes, 30 minutes, 40 minutes, 50 minutes, or 1 hour. In some embodiments, the submucosal cushion created by the shear-thinning hydrogel retains at least 75% of its height after 1 hour. In some embodiments, the submucosal cushion created by the shear-thinning hydrogel retains at least 65% of its height after 1.5 hours. In certain embodiments, the submucosal cushion created by the shear-thinning hydrogel retains a higher percentage of its initial height when the concentration of Laponite® is high (e.g., at least 2 mg mL⁻¹, at least 3 mg mL⁻¹, or at least 4 mg mL⁻¹).

In certain embodiments, the shear-thinning hydrogel forms a solid gel following injection between two planes of a tissue. In certain embodiments, the shear-thinning hydrogel forms a solid gel following injection into the submucosal space. Before injecting the shear-thinning hydrogel into the submucosal space, the hydrogel remains a liquid (e.g., inside the needle used for injection (e.g., an endoscopic needle)). This allows the shear-thinning hydrogel to flow freely through the needle and allow for injection without clogging the needle. In some aspects, upon exiting the needle tip and entering the space between two planes of a tissue or a submucosal space the shear-thinning hydrogel forms a solid gel. In certain embodiments, the shear-thinning hydrogel forms a solid gel immediately after injection.

In certain embodiments, the shear-thinning hydrogel forms a stable hydrogel. In some embodiments, a stable hydrogel is evidenced by frequency sweep measurements and monitoring the storage modulus and the loss modulus. In some embodiments, comparing the storage modulus and the loss modulus from frequency sweep measurements. In certain embodiments, the shear-thinning hydrogel exhibits a storage modulus value 5-25 times higher than the loss modulus value. In some embodiments, the shear-thinning hydrogel exhibits a storage modulus value 10-19 times higher than the loss modulus value. In certain embodiments, the shear-thinning hydrogel exhibits a storage modulus value 12-17 times higher than the loss modulus value.

Under certain conditions, the shear-thinning hydrogel undergoes a gel-sol transition (i.e., a transition of the gel network to liquid state). In some instances, a gel-sol transition is evidenced by the storage modulus decreasing rapidly with an increase in strain. In certain embodiments, the shear-thinning hydrogel exhibits a critical strain of 0.1. In some embodiments, the gel-transition occurs beyond the shear-thinning hydrogel's critical strain. In some instances, the shear-thinning hydrogel undergoes a gel-sol transition dependent on concentration of Laponite®. In certain embodiments, the strain required to cause a gel-sol transition increases with an increased concentration of Laponite® (e.g., about 1 to about 6 mg mL⁻¹). In some embodiments, the shear-thinning hydrogel undergoes transition from a gel to liquid occurs between about 10 and 1200 Pa. In some embodiments, the shear-thinning hydrogel undergoes transition from a gel to liquid occurs between about 10 and 800 Pa. In some embodiments, the shear-thinning hydrogel undergoes transition from a gel to liquid occurs between about 25 and 600 Pa. In some embodiments, the shear-thinning hydrogel undergoes transition from a gel to liquid occurs between about 100 and 600 Pa.

In certain aspects, the shear-thinning hydrogel has a reversible gel-sol transition. For example, in some instances, upon increasing strain the gel undergoes a gel-sol transition resulting in the gel behaving as a liquid. Further, upon lowering strain, the gel rapid under goes a sol-gel transition (i.e., from a liquid to a solid-gel), and the gel recovers its modulus. In certain embodiments, the gel-sol transition is completely reversible. In some embodiments, the gel-sol transition is reversible even after many transitions. In certain embodiments, the shear-thinning hydrogel is capable of self-healing. For instance, the shear-thinning hydrogel demonstrates the same mechanical properties both before and after undergoing a gel-sol transition and a sol-gel transition. In some embodiments, the shear-thinning hydrogel demonstrates about the same mechanical properties both before and after undergoing a gel-sol transition and a sol-gel transition.

In certain embodiments, the shear-thinning hydrogel is non-toxic. In some embodiments, both the layered silicate and anionic polysaccharide are non-toxic. In certain embodiments, the shear-thinning hydrogel exhibits similar toxicity to normal saline. In some embodiments, the shear-thinning hydrogel are safe for animals (e.g., humans). Toxicity can be determined by a number of ways, including by histological analysis such as hematoxylin and eosin (H&E) staining after cells or tissue have been exposed to the shear-thinning hydrogel.

As described herein, an effective amount of shear-thinning hydrogel is injected into the submucosa in order to form a submucosa cushion. In some embodiments, an effective amount is an amount effective to maintain submucosa cushion with a certain volume or height for a certain time as described herein. An effective amount may be an amount effective to maintain a submucosa cushion of a certain volume (e.g., a volume greater than a cushion formed by saline) for a certain time (e.g., as long or longer than the time needed to take advantage of the submucosa cushion (e.g., as long or longer than the time required to remove a lesion)). An effective amount may be an amount effective to maintain a submucosa cushion of a certain height (e.g., a height greater than a cushion formed by saline) for a certain time (e.g., as long or longer than the time needed to take advantage of the submucosa cushion (e.g., as long or longer than the time required to remove a lesion)). In certain aspects a certain or desired volume or height is a volume or height greater than the volume or height of a cushion created by the same volume of saline. In some aspects, a certain or desired volume or height is a volume or height which is great enough to separate a lesion from the muscular tissue layer. In some aspects, a certain or desired volume or height is a volume or height which is great enough to separate the surface mucosa from the muscular tissue layer. In certain embodiments, the effective amount is between 0.1 and 5 mL of shear-thinning hydrogel. In certain embodiments, an effective amount is approximately 0.5 mL, approximately 1 mL, approximately 1.5 mL, approximately 2 mL, or approximately 3 mL of shear-thinning hydrogel. In some embodiments, the effective amount is about 0.5 mL of shear-thinning hydrogel. In some embodiments, the effective amount is about 1 mL of shear-thinning hydrogel. In certain embodiments, the effective amount is about 1.5 mL of shear-thinning hydrogel. In some embodiments, the effective amount is about 2 mL of shear-thinning hydrogel. In certain embodiments, the effective amount is about 3 mL of shear-thinning hydrogel. In some embodiments, the effective amount is about 4 mL of shear-thinning hydrogel. In certain embodiments, the effective amount is about 5 mL of shear-thinning hydrogel. In certain embodiments, the effective amount comprises between about 2 to about 5 mg mL⁻¹ Laponite® and about 0.2 wt % sodium alginate. In some embodiments, the effective amount is about 0.5 mL of shear-thinning hydrogel with a Laponite® concentration of 2 mg mL⁻¹ and about 0.2 wt % sodium alginate. In some embodiments, the effective amount is about 1 mL of shear-thinning hydrogel with a Laponite® concentration of 2 mg mL⁻¹ and about 0.2 wt % sodium alginate. In certain embodiments, the effective amount is about 1.5 mL of shear-thinning hydrogel with a Laponite® concentration of 2 mg mL⁻¹ and about 0.2 wt % sodium alginate. In some embodiments, the effective amount is about 2 mL of shear-thinning hydrogel with a Laponite® concentration of 2 mg mL⁻¹ and about 0.2 wt % sodium alginate. In certain embodiments, the effective amount is about 3 mL of shear-thinning hydrogel with a Laponite® concentration of 2 mg mL⁻¹ and about 0.2 wt % sodium alginate. In some embodiments, the effective amount is about 0.5 mL of shear-thinning hydrogel with a Laponite® concentration of 3 mg mL⁻¹ and about 0.2 wt % sodium alginate. In some embodiments, the effective amount is about 1 mL of shear-thinning hydrogel with a Laponite® concentration of 3 mg mL⁻¹ and about 0.2 wt % sodium alginate. In certain embodiments, the effective amount is about 1.5 mL of shear-thinning hydrogel with a Laponite® concentration of 3 mg mL⁻¹ and about 0.2 wt % sodium alginate. In some embodiments, the effective amount is about 2 mL of shear-thinning hydrogel with a Laponite® concentration of 3 mg mL⁻¹ and about 0.2 wt % sodium alginate. In certain embodiments, the effective amount is about 3 mL of shear-thinning hydrogel with a Laponite® concentration of 3 mg m⁻¹ and about 0.2 wt % sodium alginate.

In certain embodiments, the shear-thinning hydrogel is injected into the submucosa via a needle. In certain embodiments, the shear-thinning hydrogel is injected via an endoscopic needle. In certain embodiments, the needle is no larger than 18 gauge needle (i.e., the diameter of the needle is no larger than about 1.27 mm). In certain embodiments, the needle is no larger than 22 gauge needle (i.e., the diameter of the needle is no larger than about 0.72 mm). In some embodiments, the needle is a 25 gauge needle. In some embodiments, the needle is a 19-27 gauge needle. In some embodiments, the needle is a 19 gauge needle. In some embodiments, the needle is a 21 gauge needle. In some embodiments, the needle is a 23 gauge needle. In certain embodiments, the shear-thinning hydrogel is injected into the submucosa through a colonoscope, an endoscope, a sigmoidoscope, a cystoscope, or a ureteroscope. In certain embodiments, the shear-thinning hydrogel is injected into the submucosa via a needle through a colonoscope, an endoscope, a sigmoidoscope, a cystoscope, or a ureteroscope. In certain embodiments, the shear-thinning hydrogel is injected into the submucosa via a needle at a rate of about 0.25 mL In certain embodiments, the shear-thinning hydrogel is injected into the submucosa via a needle at a rate of about 0.1 mL In certain embodiments, the shear-thinning hydrogel is injected into the submucosa via a needle at a rate of about 0.4 mL

In certain aspects, after injecting the shear-thinning hydrogel into the submucosa and a submucosa cushion is formed, the method further comprises removing a protrusion above the submucosal lining. In some embodiments, the protrusion is caused by a mass of cells or tissue. In certain embodiments, the protrusion is abnormal and requires medical attention. In some embodiments, the protrusion is a neoplasm. In certain embodiments, the protrusion is a non-neoplastic mass. In some embodiments, the neoplasm is a benign neoplasm, a pre-malignant neoplasm, or a malignant neoplasm. In certain embodiments, the neoplasm or non-neoplastic mass is a lesion. In certain embodiments, the method is used to remove a lesion. In certain embodiments, the method is used to remove a polyp. In certain embodiments, the polyp may be an aural polyp, a cervical polyp, a colorectal polyp, a nasal polyp, a gastric polyp, a endometrial polyp, a throat polyp, or a bladder polyp. In certain embodiments, the polyp is a malignant polyp, an adenomatous poly, a hyperplastic polyp, a hamartomatous polyp, a sessile serrated polyp, or an inflammatory polyp. In certain embodiments, the polyp is ≥10 mm. In some embodiments, the polyp is ≤10 mm. In some embodiments, the polyp is ≤5 mm. In some embodiments, the polyp is ≤1 mm.

In certain embodiments, the submucosal cushion is formed in submucosa located in the gastrointestinal, respiratory, or genitourinary tract. In some embodiments, the submucosal cushion is formed in submucosa located in the esophagus, stomach, small intestine, large intestine, pharynx, larynx, trachea, bronchi, bladder, or uterus. In certain embodiments, the submucosal cushion is formed in submucosa located in the gastrointestinal tract. In certain embodiments, the submucosal cushion is formed in submucosa located in the small intestine. In certain embodiments, the submucosal cushion is formed in submucosa located in the large intestine. In certain embodiments, the submucosal cushion is formed in submucosa located in the colon.

In some embodiments, the neoplasm is a malignant neoplasm. In certain embodiments, the malignant neoplasm is caused by cancer. In some embodiments, the cancer is esophageal cancer, stomach cancer, small bowel cancer, colon cancer, rectum cancer, throat cancer, tracheal cancer, bladder cancer, or endometrial cancer. In some embodiments, the cancer is colon cancer.

In certain embodiments, the shear-thinning hydrogel is prepared before injection. In some embodiments, prior to injection, the shear-thinning hydrogel is prepared by dissolving sodium alginate in water and adding Laponite® at the desired concentrations of each component. In certain embodiments, after preparing the shear-thinning hydrogel, the solution is sonicated. In some embodiments, after sonication, the shear-thinning hydrogel is ready for injection and injected to form a submucosal cushion.

Additional methods are provided. In some aspects, the disclosure provides a method of removing a lesion comprising injecting into the submucosa under the lesion an effective amount of a shear-thinning hydrogel, and resecting the lesion. Also provided are methods of treating cancer comprising injecting into the submucosa under a tumor an effective amount of a shear-thinning hydrogel and resecting the tumor. Further provided is a shear-thinning hydrogel for use in removing a lesion comprising injecting into the submucosa under the lesion an effective amount of a shear-thinning hydrogel and resecting the lesion. Also provided is a shear-thinning hydrogel for use in removing a tumor comprising injecting into the submucosa under the tumor an effective amount of a shear-thinning hydrogel and resecting the tumor. The effective amount of the shear-thinning hydrogel and composition of the shear-thinning hydrogel are as described herein. The disclosure further provides a shear-thinning hydrogel for use in treating cancer comprising injecting into the submucosa under a tumor an effective amount of a shear-thinning hydrogel and resecting the tumor. The effective amount of the shear-thinning hydrogel and composition of the shear-thinning hydrogel are as described herein. The cancer and tumor are also as described herein.

In some aspects, the disclosure provides a method of removing a lesion comprising injecting into the submucosa under the lesion an effective amount of a shear-thinning hydrogel comprising sodium alginate and Laponite®, and resecting the lesion. Also provided are methods of treating cancer comprising injecting into the submucosa under a tumor an effective amount of a shear-thinning hydrogel comprising sodium alginate and Laponite® and resecting the tumor. Further provided is a shear-thinning hydrogel for use in removing a lesion comprising injecting into the submucosa under the lesion an effective amount of a shear-thinning hydrogel comprising sodium alginate and Laponite® and resecting the lesion. Also provided is a shear-thinning hydrogel for use in removing a tumor comprising injecting into the submucosa under the tumor an effective amount of a shear-thinning hydrogel comprising sodium alginate and Laponite® and resecting the tumor. The effective amount of the shear-thinning hydrogel and composition of the shear-thinning hydrogel are as described herein. The disclosure further provides a shear-thinning hydrogel for use in treating cancer comprising injecting into the submucosa under a tumor an effective amount of a shear-thinning hydrogel comprising sodium alginate and Laponite® and resecting the tumor. The effective amount of the shear-thinning hydrogel and composition of the shear-thinning hydrogel are as described herein. The cancer and tumor are also as described herein.

In certain embodiments, the shear-thinning hydrogel is injected during a medical exam. In some embodiments, the shear-thinning hydrogel is injected during a colonoscopy, an endoscopy, a sigmoidoscopy, a cystoscopy, or a ureteroscopy. In certain embodiments, during the medical exam (e.g., colonoscopy, an endoscopy, a sigmoidoscopy, a cystoscopy, or a ureteroscopy), a submucosal cushion is formed by injecting the shear-thinning hydrogel into the submucosa space and a removing a protrusion (e.g., lesion, polyp, or tumor) above the cushion (e.g., resecting a lesion, polyp, or tumor above the cushion). In certain embodiments, during the medical exam (e.g., colonoscopy, an endoscopy, a sigmoidoscopy, a cystoscopy, or a ureteroscopy), tissue layers are separated by injecting the shear-thinning hydrogel between the tissue layers to form a cushion and a removing a protrusion (e.g., lesion, polyp, or tumor) above the cushion (e.g., resecting a lesion, polyp, or tumor above the cushion).

### Kits

Also encompassed by the disclosure are kits (e.g., packs). The kits provided may comprise a layered silicate, an anionic polysaccharide, and optionally, solvent. In some embodiments, the kit comprises Laponite®, sodium alginate, and optionally, water.

In certain embodiments, the kits comprise the components as described herein in a container (e.g., a vial, ampule, bottle, syringe, and/or dispenser package, or other suitable container). In some embodiments, provided kits may optionally further include a second container wherein each component in the kit is in a separate container. In some embodiments, a layered silicate described herein is provided in a first container and an anionic polysaccharide described herein is provided in a second container, wherein the first and second container are combined to form a shear-thinning hydrogel. In some embodiments, a layered silicate described herein is provided in a first container, an anionic polysaccharide described herein is provided in a second container, and a solvent is provided in a third container, wherein the first, second, and third containers are combined to form a shear-thinning hydrogel. In some embodiments, Laponite® is provided in a first container and sodium alginate is provided in a second container, wherein the first and second container are combined to form a shear-thinning hydrogel. In some embodiments, Laponite® is provided in a first container, sodium alginate is provided in a second container, and water in a third container, wherein the first, second, and third containers are combined to form a shear-thinning hydrogel. In certain embodiments, the kits further comprise instructions for forming a shear-thinning hydrogel from the kit components. In certain embodiments, the kits further comprise devices for delivering the shear-thinning hydrogel to the site of use (e.g., the site of cushion formation or tissue separation) such as a syringe, needle (e.g., endoscopic needles), and/or a catheter.

Thus, in one aspect, provided are kits are useful for forming a submucosa cushion. In some embodiments, the kits are useful for removing a protrusion (e.g., from above the submucosal lining). In some embodiments, the kits are useful for removing a lesion (e.g., from above the submucosal lining). In some embodiments, the kits are useful for removing a tumor (e.g., from above the submucosal lining). In some embodiments, the kits are useful for removing a polyp (e.g., from above the submucosal lining). In some embodiments, the kits are useful for treating polyps (e.g., by removing a polyp from above the submucosal lining). In some embodiments, the kits are useful for treating cancer (e.g., by removing a tumor from above the submucosal lining). In some embodiments, the kits are useful for treating lesions (e.g., by removing a lesion from above the submucosal lining).

In certain embodiments, a kit described herein further includes instructions for using the kit. The instructions may include instructions for forming the shear-thinning hydrogel. In some embodiments, the kit includes instructions for injecting the shear-thinning hydrogel. In some embodiments, the kit includes instructions for using the shear-thinning hydrogel to form a submucosal cushion. In certain embodiments, the kit includes instructions for removing a protrusion, lesion, tumor, or polyp.

A kit described herein may also include information as required by a regulatory agency. In certain embodiments, the information included in the kits is usage information.

### EXAMPLES

In order that the present disclosure may be more fully understood, the following examples are set forth. The synthetic and biological examples described in this Application are offered to illustrate the compounds, pharmaceutical compositions, methods, and uses provided herein and are not to be construed in any way as limiting their scope.

### Results and Discussion

### Design and Preparation of EISHs

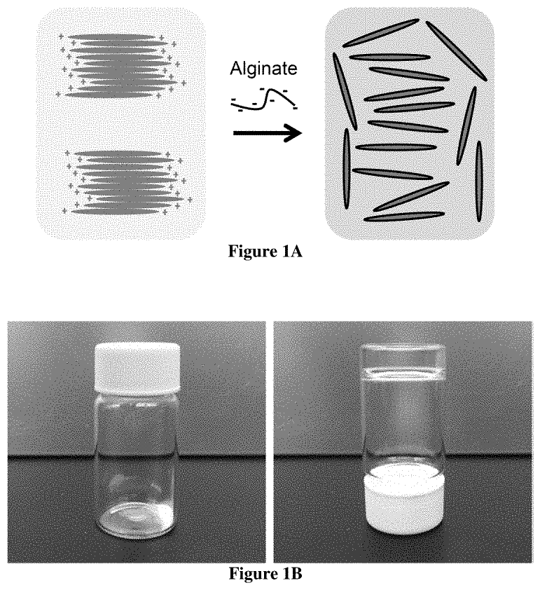

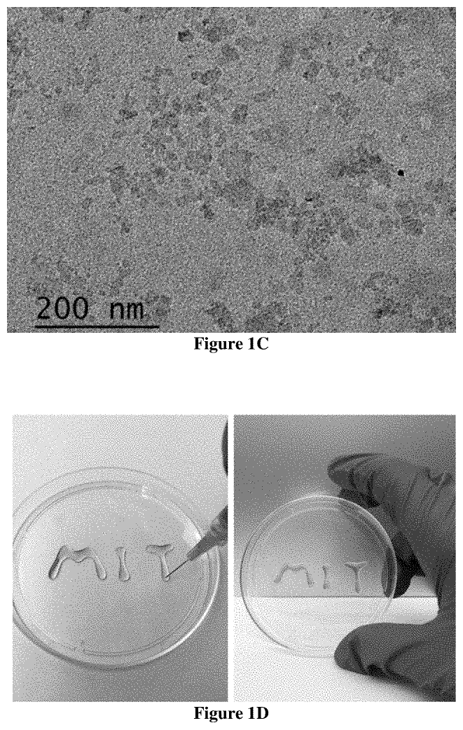

Shear-thinning is a term used in rheology to describe non-Newtonian fluids which demonstrate viscous flow under shear stress and subsequent recovery upon removal of the stress.^{[28]} Recognizing this property, it was hypothesized that shear-thinning hydrogels could serve as a platform for endoscopic injection and cushion formation. Laponite®, a layered nanosilicate with good biocompatibility and biodegradability, which is commonly utilized as a rheology modifier and additive to promote shear-thinning and thixotropic behavior was recognized as a material candidate for the synthesis of the gels.^{[29,30]} Alginate, an anionic polysaccharide extracted from seaweeds, is biocompatible, and its aqueous solutions demonstrate non-Newtonian fluid behavior with shear-thinning properties.^{[31]} Described herein, alginate was used to exfoliate and disperse Laponite® nanosheets by mutual repulsion resulting from a possible site-specific wrapping of their positive-charged edge parts^{[29]} with anionic alginate (FIG. 1A). Laponite® was briefly exfoliated in 0.2 wt % sodium alginate aqueous solution and a transparent EISH was formed immediately after sonication (FIG. 1B). Transmission electron microscopy (TEM) images show that Laponite® nanosheets were dispersed homogeneously (FIG. 1C). The prepared EISHs could be easily injected through a 25-gauge needle and immediately reformed a solid gel after injection, as shown in FIG. 1D.

### Rheological Properties of EISHs

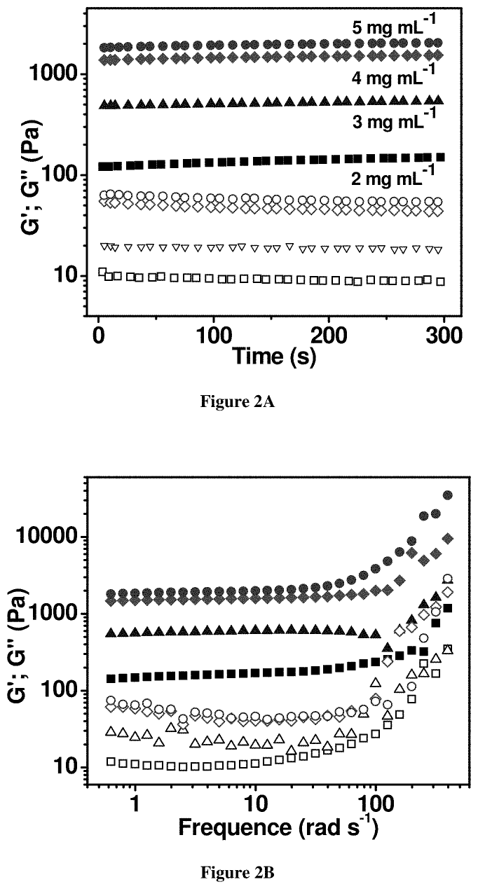

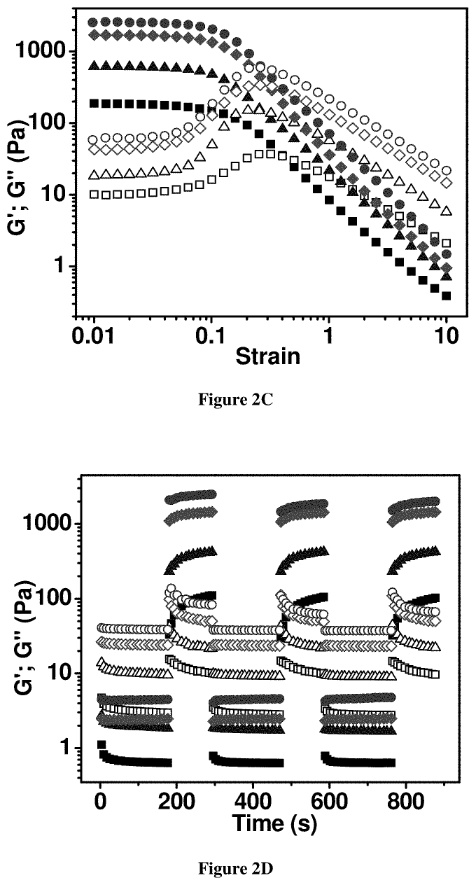

The effect of Laponite® concentration on the rheological behavior of EISHs was investigated. Oscillatory measurements indicated that both the storage modulus (G′) and loss modulus (G″) of EISHs increased with concentration. Time sweep experiments showed that the G′ and G″ values of EISHs separately increased from ˜10 and ˜100 Pa to ˜70 and ˜1100 Pa with increasing Laponite® concentration from 2 to 5 mg mL⁻¹ (FIG. 2A). Frequency sweep measurements displayed that EISHs exhibited constant G′ values approximately 10-19 times higher than G″ values throughout the frequency sweep from 0.1 to 100 rad s⁻¹, indicating the formation of stable hydrogels (FIG. 2B). Strain-dependent oscillatory rheology experiments were further performed to examine the linear viscoelastic range of EISHs. As shown in FIG. 2C, the moduli of EISHs were independent of strain amplitude and showed linear viscoelastic behavior at low strain ranging from 0.01 to 0.1. Beyond their critical strains around 0.1, the G′ values of EISHs decreased rapidly with the increase of strain, suggesting the gels underwent gel-sol transition and behaved as liquids. The cross point of G′ and G″, representing the transition of the gel network to a liquid state (solution behavior: G′<G″, solid behavior: G′>G″), increased from ˜25 to ˜600 Pa with increasing Laponite® concentration from 2 to 5 mg mL⁻¹, respectively. These data demonstrated that the rheological behavior and shear-thinning properties of EISHs can be conveniently tuned by adjusting Laponite® concentrations.

Step-strain measurements were performed to verify the reversible gel-sol transition of EISHs. The deformation and recovery of EISHs were conducted at repeated cycles of 3 min low magnitude strain of 0.5% and 2 min high magnitude strain of 500% oscillations at 6.3 rad s⁻¹. After applying alternative low and high strains, the moduli of EISHs during the strain changes was monitored. As shown in FIG. 2D, the gels underwent gel-sol transition and behaved as liquids upon increasing oscillatory strain from 0.5% to 500%. Inversely, EISHs rapidly underwent sol-gel transition and recovered back to their initial moduli immediately with lowering the strain from 500% to 0.5%. The gel-sol transition was reversible and all gels were capable of self-healing to their original state without showing any signs that mechanical fidelity was compromised, irrespective of the number of times they were previously shear-thinned. These data demonstrated the robust reversibility of the mechanical properties of EISHs.

### In Vitro Evaluation of EISHs

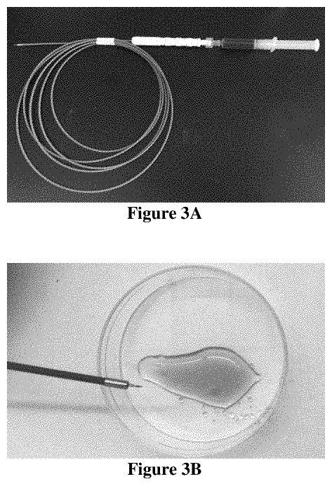

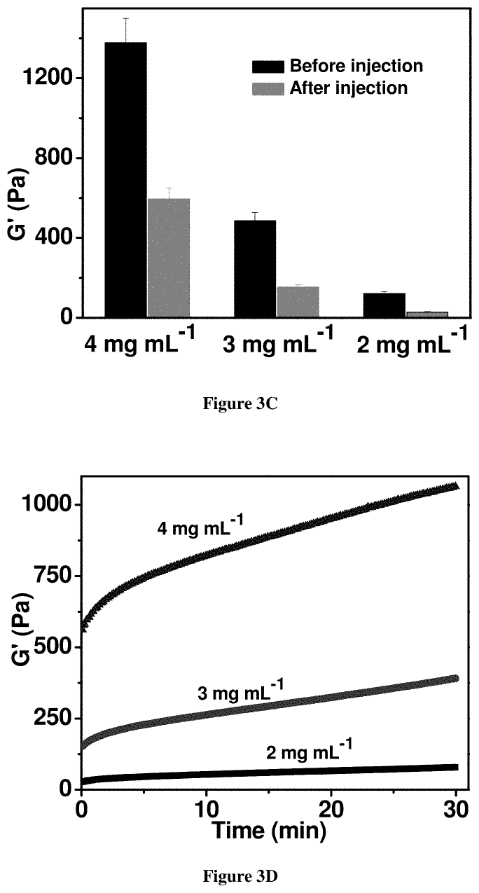

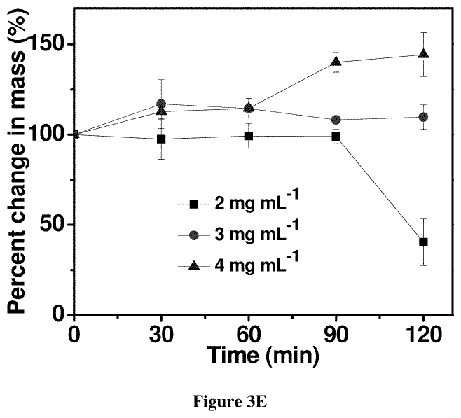

Next, the injection feasibility of EISHs was studied by utilizing a standard 25-gauge endoscopic needle ^{[32]} that is widely used for in vivo submucosal injection in endoscopic procedures (FIG. 3A). Representative formulations of EISHs with a Laponite® concentration of 2 mg mL⁻¹ could be injected as shown in FIG. 3B. The storage modulus of EISHs with Laponite® concentrations of 2, 3 and 4 mg mL⁻¹ decreased to 23%, 31% and 43% respectively, after passing through a 25-gauge needle with an injection speed of 0.25 mL (FIG. 3C). To elucidate the recovery capability of EISHs, oscillatory time sweep rheology measurements were performed immediately following the injection. As shown in FIG. 3D, the modulus of EISHs with Laponite® concentrations of 2, 3 and 4 mg mL⁻¹ increased by 2.9, 2.6 and 1.9 times in 30 min, respectively. These results demonstrate the feasibility of injection of EISHs and their rapid conversion to a solid gel following injection.

The stability of EISHs was evaluated by measuring their erosion kinetics in a physiological environment. A volume of 0.5 mL of EISHs were injected in saline and further incubated at 37° C. for predetermined time intervals. The volume of remaining gels at each time point was recorded to calculate the erosion kinetics of EISHs. As shown in FIG. 3E, the volume of EISHs with Laponite® concentration of 2 mg mL⁻¹ remained constant within 1.5 h. While the volume of the gels decreased to 40% with further prolonging incubation time to 2 h, which could be explained by the passive diffusion of both Laponite® and alginate. However, EISHs with a higher Laponite® concentration of 3 mg mL⁻¹ maintained their volume up to 2 hours. Interestingly, EISHs with a high concentration of 4 mg mL⁻¹ swelled gradually and reached to 1.4 times their initial volume after 2 hours incubation. It was speculated that the dispersion of a high Laponite® content of 4 mg mL⁻¹ in alginate aqueous solution forms steady hydrogels that can promote their water absorption. These erosion profiles suggested the potential of EISHs to resist passive diffusion and achieve relative long-term submucosal cushions.

### Endoscopic Development of Submucosal Cushions

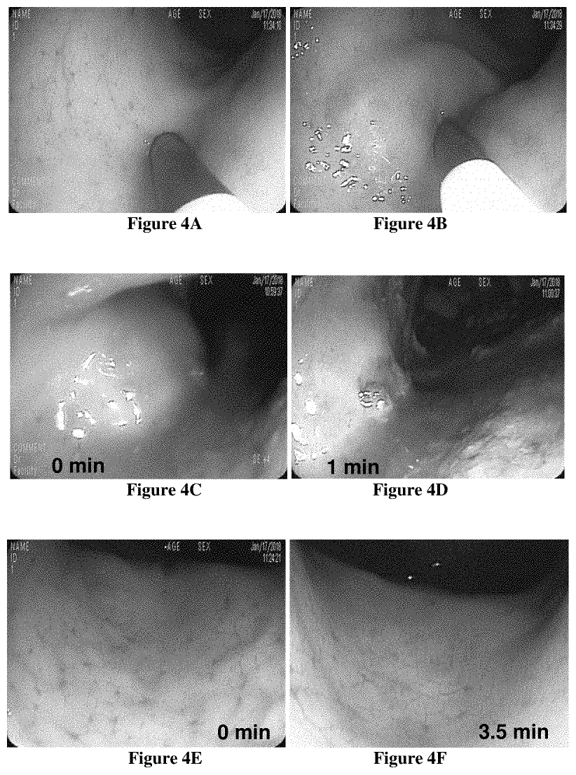

Having confirmed the feasible injection and rapid recovery as well as high stability of EISHs, their performances for cushion development in vivo was tested. Yorkshire pigs weighing 40-80 kg were used as a large animal model and endoscopic injection was utilized to develop submucosal cushions in the colon. As displayed in FIGS. 4A and 4B, a clear cushion was easily formed by submucosal injection of 1.5 cc of EISHs (2 mg mL⁻¹) through an endoscopic needle. Four different injections were performed and each time a well-formed cushion was observed. Additionally, endoscopic videography was used to observe the duration of cushions formed by EISHs. The cushions created by normal saline flattened dramatically within 1 min (FIGS. 4C and 4D), while the cushions produced by EISHs remained almost unchanged for up to 3.5 minutes (FIGS. 4E and 4F), showing the prolonged duration of cushions developed by these gels.

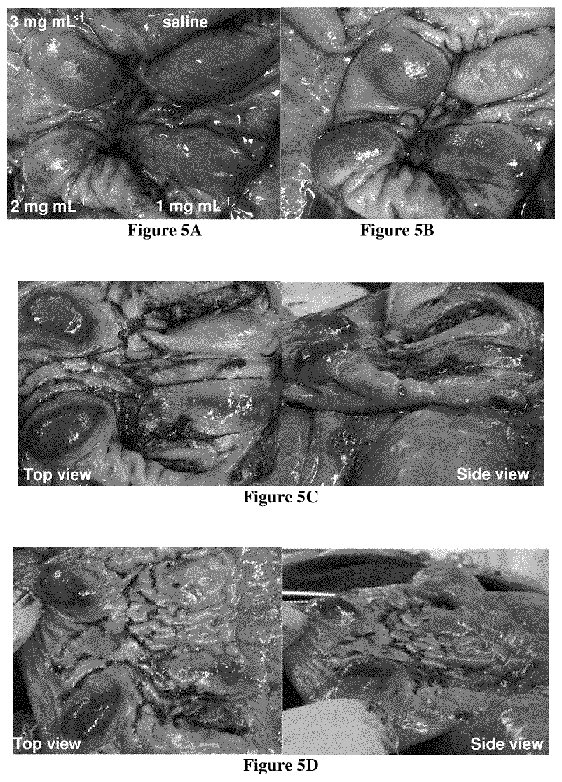

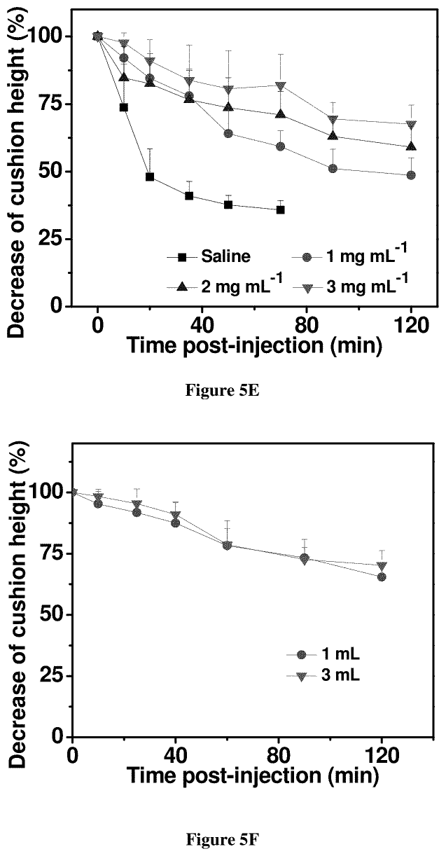

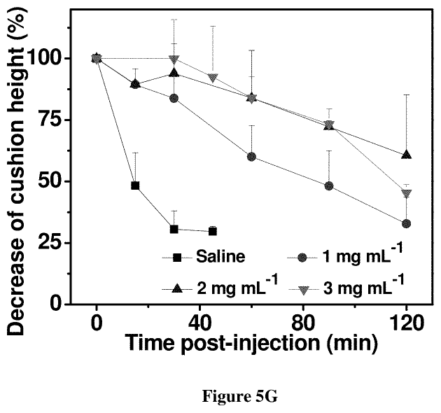

To accurately evaluate the duration of cushions, their heights were measured by a digital caliper through a midline laparotomy during a terminal procedure. Two cc of EISHs with different Laponite® concentrations were submucosally injected in pig colon and the heights of the resulted cushions were measured at predetermined time intervals. Normal saline was used as a control. As observed in FIGS. 5A to 5D, with the increase of incubation time, the heights of cushions created by EISHs decreased at a far slower rate than that of saline. At 20 min incubation, the height of cushions developed by saline decreased dramatically to less than 50%, while the heights of all cushions created by EISHs with different concentrations from 1 to 3 mg mL⁻¹ remained as high as 82% to 91% (FIG. 5E). Even with incubation time prolonging to 2 hours, the heights of cushions elevated by EISHs with a low Laponite® concentration of 1 mg mL⁻¹ remained around 50%. The cushions developed by EISHs with a Laponite® concentration of 3 mg mL⁻¹ maintained up to 68% of their initial heights after 2 hours incubation, demonstrating the prolonged durations. The effect of injection volume on the duration of cushions was also investigated. As illustrated in FIG. 5F, with increasing injection volume from 1 to 3 mL, no obvious differences were observed between the heights of cushions produced by EISHs with a Laponite® concentration of 2 mg mL⁻¹. The durations of cushions elevated in pig small intestine were also investigated. As expected, their durations were quite similar to those created in pig colon (FIG. 5G), showing the capabilities of EISHs to develop durable submucosal cushions at different sites.

### In Vivo Toxicity of EISHs

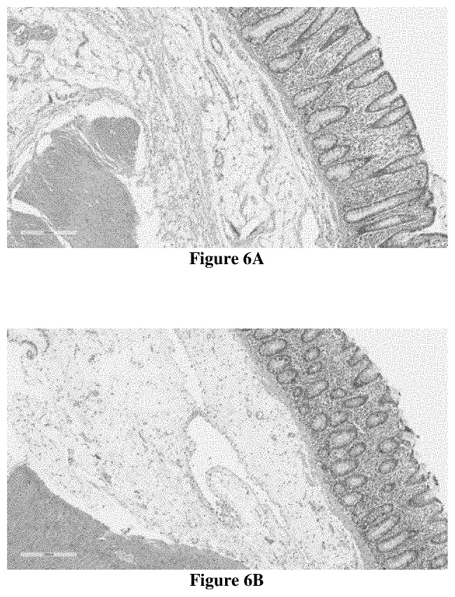

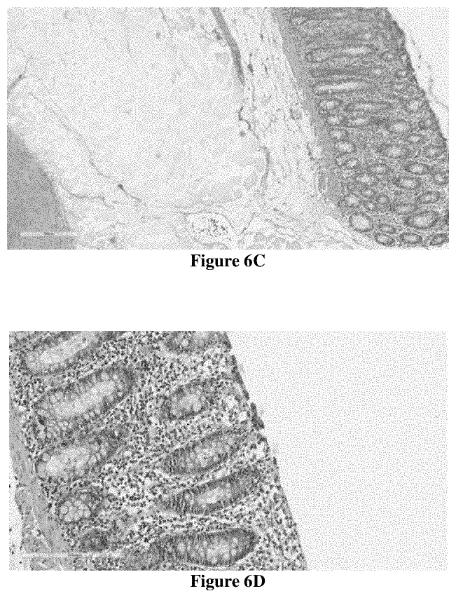

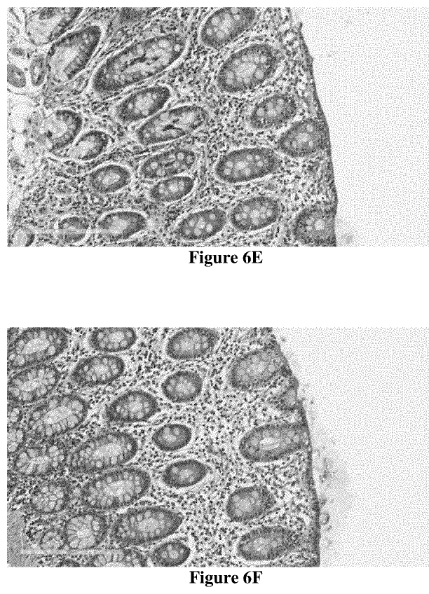

To complete the exploration of the advantages of EISHs as submucosal injection agents, their in vivo toxicity by histological analysis (33) was analyzed. Hematoxylin and eosin (H&E) staining was used to evaluate the toxicity of EISHs against the tissue of pig colon in vivo. Three cc of EISHs with a Laponite® concentration of 3 mg mL⁻¹ was submucosally injected to the colon of a sedated pig and normal saline was used as a control. At 2 hours post-injection, the pig was euthanized, and the tissues were immediately harvested, fixed by formalin and further embedded by paraffin. The resultant tissues were then sectioned and stained by H&E for confocal microscope imaging. As shown in FIGS. 6A to 6C, no significant difference was observed between the tissues treated by EISHs and the control tissues injected with normal saline. Similar results were obtained by incubation of EISHs on the top of the mucus for 2 hours (FIGS. 6D to 6F), demonstrating the low in vivo toxicity of these gels using as cushion development agents.

### Conclusions

In summary, reported herein is the development and application of shear-thinning hydrogels as safe and endoscopically injectable solutions capable of establishing durable submucosal cushions. It was shown that these shear-thinning hydrogels can be rapidly prepared by dispersing commercially available Laponite® into an aqueous solution of alginate and their rheological properties can be easily tuned by varying the concentrations of Laponite®. Also, these hydrogels can be injected through a standard endoscopic needle and further demonstrate their low toxicity as well as the enhanced durations of cushions elevated by these gels. In summary, the hydrogel materials developed herein present 1) commercially available and inexpensive resources; 2) tunable shear-thinning properties and endoscopically injectable capability; and 3) good biocompatibility and improved stability for the development of durable submucosal cushions. All these features make EISHs a promising set of hydrogel materials for broad application in mucosal resection techniques and potentially luminal constriction, drug delivery, and tissue engineering.

### Materials and Methods

### Materials

Sodium alginate, Laponite®, Indigo carmine, methylene blue, and other chemical reagents were purchased from Sigma and used as received unless otherwise noted. Nanopure water (18 MΩ cm) was acquired by means of a Milli-Q water filtration system, Millipore (St. Charles).

### Transmission Electron Micrograph (TEM) Measurements

TEM experiments were carried out on a JEOL 2100 FEG instrument at an acceleration voltage of 200 kV. The TEM sample was prepared by dropping the exfoliated Laponite® solutions onto a 300-mEISH carbon-coated copper grid. Samples were blotted away after 30 min incubation at the room temperature and then washed twice with distilled water and air dried prior to imaging.

### Preparation of EISHs.

0.2% sodium alginate aqueous solution was prepared as stock solution. Laponite® was added into the stock solution with various concentrations and then sonicated for ˜2-5 minutes to obtain EISHs. EISHs with Laponite® concentrations of 2 mg mL⁻¹, 3 mg mL⁻¹, 4 mg mL⁻¹ and 5 mg mL⁻¹ were prepared accordingly and used directly for further measurements.

### Measurements of the Rheological Properties of EISHs

Dynamic oscillatory time, frequency and strain sweeps were performed using an AR2000 stress-controlled rheometer (TA Instruments, New Castle, Del.) with 25 mm steel plate geometry at a 27 mm gap distance. Laponite® was dispersed in 0.2 wt % alginate solution by sonication to form EISHs with specified compositions and the gels were applied between the 2 plates of the rheometer. The top plate was lowered to a 27 mm gap distance and excess gel was scraped off. Care was taken to achieve a homogeneous distribution of gel within the top and bottom plates of the rheometer. Dynamic oscillatory time sweeps were collected at angular frequencies of 6.3 rad s⁻¹ and 0.5% strain. An initial strain amplitude sweep was performed at 25° C. at different frequencies to determine the linear viscoelastic range for the gels. Rheological properties were examined by frequency sweep experiments at fixed strain amplitude of 0.5%. Experiments were repeated on 3 to 4 samples and representative data is presented. For shear recovery experiments at 6.3 rad s⁻¹, shear thinning was induced via application of 500% strain for 2 min. The strain was released to 0.5% for 3 min to allow the gel to recover.

### Erosion Studies of EISHs

The erosion kinetics of the EISHs was measured in a physiological environment. A volume of 0.5 mL of EISHs were injected in saline and further incubated at 37° C. for 30 min, 60 min, 90 min and 120 min, respectively. The volume of remaining gels at each time point was recorded to calculate the erosion kinetics of EISHs.

### Ex Vivo Cushion Development in Pig Colon

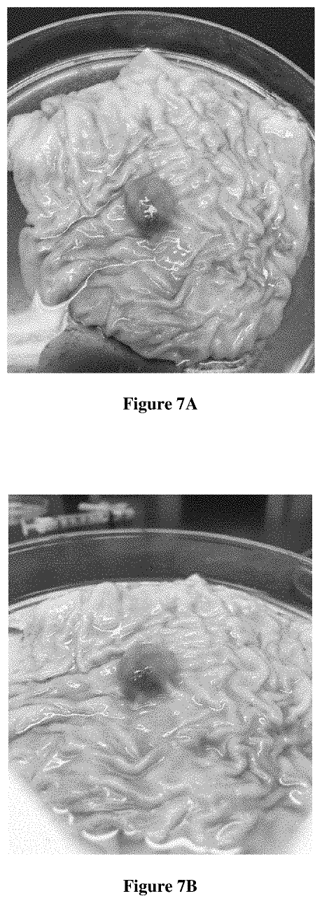

Ex vivo cushion development was performed by injection of 0.5 cc EISHs (2 mg mL⁻¹) into the pig colon. The colon tissue was isolated from freshly procured intact gastrointestinal tracts from pigs from selected local slaughter houses. The top view and the side view of the developed cushions were shown in FIGS. 7A and 7B.

### In Vivo Cushion Development in a Pig Model

All pig experiments were approved by the Committee on Animal Care at the Massachusetts Institute of Technology. Female Yorkshire pigs (40-80 kg) were obtained from Tufts University and housed under conventional conditions. Animals were randomly selected for the experiments. The animals were placed on a liquid diet for 24 hours prior to the experiment with the morning feed held on the day of the experiment. At the time of the experiment, the pigs were anesthetized with intramuscular administration of Telazol® (tiletamine/zolazepam 5 mg kg⁻¹), xylazine (2 mg kg⁻¹) and atropine (0.04 mg kg⁻¹). An endoscope (Pentax,US endoscopy) was inserted into the distal colon and a Can-Locke needle was inserted through the channel of the endoscope into the colon. Subsequently, 1.5 mL of saline and hydrogel were separately injected into the submucosal space, repeated 3 times. Videos were recorded to monitor the decrease of the size of the cushions lift. All animals were recovered from anesthesia.

Measurements of In Vivo Cushion Duration.

All procedures were conducted in accordance with protocols approved by the Massachusetts Institute of Technology Committee on Animal Care. Female Yorkshire swine, approximately 40-80 kg in body weight were anesthetized with intramuscular administration of Telazol® (tiletamine/zolazepam 5 mg kg⁻¹), xylazine (2 mg kg⁻¹) and atropine (0.04 mg kg⁻¹). Animals were intubated and maintained on 2-3% isoflurane in oxygen. As part of a terminal or non-survival procedure, a midline laparotomy was performed and the proximal jejunum or distal colon were accessed and stabilized with gauze. A longitudinal incision was made to access the luminal side and 2 cc normal saline solution and 1 mg mL⁻¹ EISH, 2 mg mL⁻¹ EISH, and 3 mg mL⁻¹ EISH were injected into the submucosal space. to form the cushions. The length, width and the height of the cushions were measured at 0, 30, 60, and 120 min after the injection. 1 mL, 2 mL and 3 mL 2 mg mL⁻¹ EISHs were also injected to investigate the cushion properties. Animal were euthanized prior to anesthetic recovery with intravenous administration of 120 mg kg⁻¹ of sodium pentobarbital.

### H & E Staining

The toxicity of EISHs were evaluated during an in vivo terminal experiment. All procedures were conducted in accordance with protocols approved by the Massachusetts Institute of Technology Committee on Animal Care. Pigs were intubated and maintained on 2-3% isoflurane in oxygen. A midline laparotomy was performed and the proximal jejunum accessed and stabilized with gauze. Three cc normal saline solution and 3 mg mL⁻¹ EISH were submucosally injected to the pig colon to form the cushions. Meantime, multiple 4-5 cm incisions were made along the anitmesenteric side of the colon. Three cc normal saline solution and 3 mg mL⁻¹ EISH were incubated on the top of the mucus using wells secured with carbopol and covered with an adhesive membrane. The pigs were euthanized with sodium pentobarbital (120 mg kg) intravenously prior to tissue collection. Tissues were harvested and placed into formalin (4%). After tissues were fixed in formalin, they were paraffin embedded, sectioned and stained with hematoxylin and eosin for analysis.

### REFERENCES

[1] D. A. Joseph, R. G. S. Meester, A. G. Zauber, D. L. Manninen, L. Winges, F. B. Dong, B. Peaker, M. van Ballegooijen, Cancer. 2016, 122, 2479.
[2] D. A. Corley, C. D. Jensen, A. R. Marks, W. K. Zhao, J. K. Lee, C. A. Doubeni, A. G. Zauber, J. de Boer, B. H. Fireman, J. E. Schottinger, V. P. Quinn, N. R. Ghai, T. R. Levin, C. P. Quesenberry, N Engl J Med 2014, 370, 1298.
[3] D. A. Corley, C. D. Jensen, A. R. Marks, W. K. Zhao, J. de Boer, T. R. Levin, C. Doubeni, B. H. Fireman, C. P. Quesenberry, Clin Gastroenterol Hepatol. 2013, 11, 172.
[4] H. Ladhani, H. Khadra, J. Marks, (eds) Advanced Colonoscopy and Endoluminal Surgery. Springer, Springer, Cham. 2017.
[5] H. Yandrapu, M. Desai, S. Siddique, P. Vennalaganti, S. Vennalaganti, S. Parasa, T. Rai, V. Kanakadandi, A. Bansal, M. Titi, A. Repici, M. L. Bechtold, P. Sharma, A. Choudhary, Gastrointest Endosc. 2017, 85, 693.
[6] M. Tada, M. Shimada, F. Murakami, M. Mizumachi, K. Arima, H. Yanai, S. Oka, M. Shigeeda, T. Aibe, Y. Okazaki, T. Takemoto, M. Murata, M. Ogino, Z. Kinoshita, K. Kinoshita, Y. Lida, Gastroenterol Endosc. 1984, 26, 833.
[7] A. Repici, M. Wallace, P. Sharma, P. Bhandari, G. Lollo, R. Maselli, C. Hassan, D. K. Rex, Gastrointest Endosc. 2018, 88, 527.
[8] N. Mehta, A. T. Strong, M. Franco, T. Stevens, R. Chahal, S. Jang, R. Lopez, D. Patil, S. Abe, Y. Saito, T. Uraoka, J. Vargo, A. Bhatt, Digest Endosc. 2018, 30, 347.
[9] Y. S. Jung, D. Il Park, Gastrointest Interv. 2013, 2,73.
[10] A. B. Feitoza, C. J. Gostout, L. J. Burgart, A. Burkert, L. J. Herman, E. Rajan, Gastrointest Endosc. 2003, 57, 41.
[11] A. Moss, M. J. Bourke, A. J. Metz, Am J Gastroenterol. 2010, 105, 2375.
[12] A. O. Ferreira, J. Moleiro, J. Tones, M. Dinis-Ribeiro, Endoscopy International Open. 2016, 4, E1.
[13] M. Ishihara, I. Kumano, H. Hattori, S. Nakamura, J Artif Organs. 2015, 18, 191.
[14] T. Uraoka, Y. Saito, K. Yamamoto, T. Fujii, Drug Des Dev Ther. 2008, 2, 131.
[15] P. Katsinelos, J. Kountouras, G. Paroutoglou, G. Chatzimavroudis, C. Zavos, I. Pilpilidis, G. Gelas, D. Paikos, K. Karakousis, Gastrointest Endosc. 2008, 68, 692.
[16] M. Fujishiro, N. Yahagi, K. Kashimura, Y. Mizushima, M. Oka, S. Enomoto, N. Kakushima, K. Kobayashi, T. Hashimoto, M. Lguchi, Y. Shimizu, M. Ichinose, M. Omata. Endoscopy. 2004, 36, 579.
[17] M. Yamasaki, K. Kume, I. Yoshikawa, M. Otsuki, Gastrointest Endosc. 2006, 64, 958.
[18] L. Lenz, V. D. Sena, F. S. Nakao, G. P. de Andrade, M. R. da S. Rohr, A. P. Ferrari Jr, Arq Gastroenterol. 2010, 47, 184.
[19] Y. Matsui, M. Inomata, K. Izumi, K. Sonoda, N. Shiraishi, S. Kitano, Gastrointest Endosc. 2004, 60, 539.
[20] S. H., J. HoonPark, D. HyunPark, I. KwunChung, H. SooKim, S. HeumPark, S. JooKim, H. DeukCho, Gastrointest Endosc. 2006, 64, 691.
[21] Y. Shastri, S. Kriener, W. F. Caspary, A. R. J. Schneider, Scand J Gastroenterol. 2007, 42, 1369.
[22] T. R. Soong, J. Nayor, M. D. Stachler, M. Perencevich, K. Jajoo, J. R. Saltzman, N. I. Lindeman, A. Srivastava, Modern Pathology. 2018, 1.
[23] S. Jin Jeon, A. W. Hauser, R. C. Hayward, Acc. Chem. Res. 2017, 50, 161.
[24] A. S. Hoffman, Adv Drug Deliver Rev. 2012, 64, 18.
[25] M. H. Chen, L. L. Wang, J. J. Chung, Y. Hun Kim, P. Atluri, J. A. Burdick, ACS Biomater Sci Eng. 2017, 3, 3146.
[26] L. S. Nair, C. T. Laurencin, M. Tandon, Advanced Biomaterials: Fundamentals, Processing, and Applications. 2010.
[27] P. J. Kondiah, Y. E. Choonara, P. P. D. Kondiah, T. Marimuthu, P. Kumar, L. C. Du Toit, V. Pillay, Molecules. 2016, 21, 1580.
[28] M. Guvendiren, H. D. Lu, J. A. Burdick, Soft Matter. 2012, 8, 260.
[29] Q. Wang, Justin L. Mynar, M. Yoshida, E. Lee, M. Lee, Kou Okuro, K. Kinbara, T. Aida, Nature. 2010, 463, 339.
[30] J. L. Davila, M. A. d'Ávila, Carbohydrate Polymers. 2017, 157,1.
[31] R. A. Rezende, P. J. Bártolo, A. Mendes, R. M. Filho, J Appl Polym Sci. 2009, 113, 3866.
[32] E. L. A. Artifon, H. G. Guedes, S. Cheng, Gastroenterology. 2017, 153, 881.
[33] D. Polymerosa, G. Kotsalidis, K. Triantafyllou, G. Karamanolis, J. G. Panagiotides, S. D. Ladas, Digest Liver Dis. 2010, 42, 226.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of forming an interlayer tissue cushion comprising injecting between two tissue layers an effective amount of a shear-thinning hydrogel.

2. The method of claim 1, wherein the interlayer cushion is submucosal cushion formed by injecting into the submucosa an effective amount of a shear-thinning hydrogel.

3. The method of claim 1, wherein the hydrogel comprises a layered silicate and an anionic polysaccharide.

4. The method of claim 3, wherein the layered silicate is a montmorillonite, saponite, hectorite, or subclasses thereof.

5. The method of claim 4, wherein the layered silicate is Laponite®.

6. The method of claim 5, wherein the concentration of Laponite® is between about 2 to about 5 mg mL⁻¹.

7. The method of claim 3, wherein the anionic polysaccharide is an algin, an arabinoxylan, a carrageenan, a furcellaran, a gellan, a gum arabic, a gum ghatti, a gum karaya, a gum tragacanth, an okra gum, a pectic acid, a pectin, a psyllium seed gum, a xanthan, a xylan, a carboxymethylcellulose, or a salt thereof.

8. The method of claim 7, wherein the anionic polysaccharide is sodium alginate.

9. The method of claim 8, wherein the concentration of sodium alginate is about 0.1 to about 1 wt %.

10. The method of claim 2, wherein the shear-thinning hydrogel comprises Laponite® and sodium alginate.

11. (canceled)

12. The method of claim 2, wherein the volume of the submucosal cushion created by the shear-thinning hydrogel is at least maintained for at least 1.5 hours.

13-14. (canceled)

15. The method of claim 2, wherein the submucosal cushion retains at least 75% of its height after 40 minutes.

16-19. (canceled)

20. The method of claim 2, wherein the shear-thinning hydrogel exhibits a storage modulus value 10-19 times higher than the loss modulus value.

21. (canceled)

22. The method of claim 2, wherein the shear-thinning hydrogel undergoes transition from a gel to liquid occurs between about 25 and 600 Pa.

23-32. (canceled)

33. The method of claim 2 further comprising removing a protrusion above the submucosal lining.

34. (canceled)

35. The method of 33, wherein the protrusion is a neoplasm or non-neoplastic mass.

36-40. (canceled)

41. The method of claim 2, wherein submucosa is located in the gastrointestinal, respiratory, or genitourinary tract.

42-44. (canceled)

45. A method of removing a lesion comprising injecting into the submucosa under the lesion an effective amount of a shear-thinning hydrogel and resecting the lesion.

46. A method of preventing or treating cancer comprising injecting into the submucosa under a tumor an effective amount of a shear-thinning hydrogel and resecting the tumor.

47-50. (canceled)

51. A kit comprising:
Laponite®;
sodium alginate; and
optionally, water.

52-55. (canceled)
